(19) United States
(12) Patent Application Publication
(10) Pub. No.: US 2026/0108675 A1
(43) Pub. Date: Apr. 23, 2026

(54) SYSTEMS AND METHODS FOR DETECTING STATE OF A MEDICATION DELIVERY DEVICE

(71) Applicants: Eli Lilly and Company, Indianapolis, IN (US); Identiv, Inc., Santa Ana, CA (US)

(72) Inventors: Thomas Christian GERMANN, Aschau im Chiemgau (DE); Douglas James GORR, Indianapolis, IN (US); Jeffrey Dirk LOVELACE, Tryon, NC (US); Edward Joseph MORRIS, Bloomington, IN (US); Louis Stevens SOMLAI, Carmel, IN (US)

(21) Appl. No.: 19/111,409

(22) PCT Filed: Sep. 14, 2023
(86) PCT No.: PCT/US2023/074127

§ 371 Date: Mar. 13, 2025

Related U.S. Application Data
(60) Provisional application No. 63/406,754, filed on Sep. 15, 2022.

(51) Int. Cl. A61M 5/20

(52) U.S. Cl. CPC A61M 5/2033; A61M 2205/13; A61M 2205/3317; A61M 2205/3368; A61M 2205/3561; A61M 2205/3576; A61M 2205/8243

(57) ABSTRACT

A sensing system for determining a state of a compression spring is provided. The system may include capacitive plates positioned on opposite sides of the compression spring, a temperature sensor, and at least one processing circuit configured to measure a capacitance between the capacitive plates, measure a temperature based on a signal output by the temperature sensor, and determine whether the compression spring is in a compressed state or an expanded state based on the measured capacitance and the measured temperature. In some embodiments, the sensing system may be used to detect a state of a compression spring disposed on or within a medication-delivery device, so as to determine a state of the medication-delivery device. For example, the sensing system may be used to determine whether the medication-delivery device has been activated, or whether the device has completed delivery of the medication.

### FIELD OF THE DISCLOSURE

The present disclosure relates to systems and methods for detecting a state of a compression spring. More particularly, the present disclosure relates to systems and methods for detecting whether a compression spring used on or within a medication-delivery device is in a compressed state or an expanded state, and to infer therefrom a state of the medication-delivery device.

### BACKGROUND OF THE DISCLOSURE

Medication-delivery devices commonly use compression springs to operate. Such springs may transition from a compressed state to an expanded state, or from an expanded state to a compressed state, when components of delivery devices move into certain positions or transition into different configurations. For example, springs may be used to drive a syringe assembly so as to penetrate a user's skin, or to pump a medication once the needle is inserted. Springs may also be used to retract the syringe assembly after use, or to extend a needle guard that surrounds an injection needle, so as to mitigate the chances of an accidental needle prick.

### SUMMARY

According to an exemplary embodiment of the present disclosure, a medication delivery device is provided for determining a state of a compression spring of the medication delivery device, the sensing system comprising: a first capacitive plate and a second capacitive plate configured such that the compression spring is disposed between at least a portion of the first capacitive plate and at least a portion of the second capacitive plate; a temperature sensor; and at least one processing circuit connected to the first capacitive plate, the second capacitive plate, and the temperature sensor, the at least one processing circuit configured to: measure a capacitance between the first capacitive plate and the second capacitive plate, measure a temperature based on a signal output by the temperature sensor, and determine whether the compression spring is in a compressed state or an expanded state based on the measured capacitance and the measured temperature.

According to another embodiment of the present disclosure, an injection system is provided, comprising: a medication-delivery device housing; a syringe assembly at least partially disposed within the medication-delivery device housing, the syringe assembly including a reservoir configured to hold a medication and an injection needle; a drive mechanism at least partially disposed within the medication-delivery device housing configured to, upon activation by a user, dispense medication from the syringe assembly via the injection needle, the drive mechanism including a compression spring that is configured to transition from a compressed state to an expanded state, or to transition from an expanded state to a compressed state, after said activation; and a sensing system comprising: a first capacitive plate and a second capacitive plate configured such that the compression spring is disposed between at least a portion of the first capacitive plate and at least a portion of the second capacitive plate; a temperature sensor, and at least one processing circuit configured to: measure a capacitance between the first capacitive plate and the second capacitive plate, and measure a temperature based on a signal output by the temperature sensor.

According to yet another embodiment of the present disclosure, a method is provided for determining a state of a compression spring positioned between at least a portion of a first capacitive plate and at least a portion of a second capacitive plate, the method comprising: measuring a temperature using a temperature sensor; measuring a capacitance between the first capacitive plate and the second capacitive plate; and determining whether the compression spring is in a compressed state or an expanded state based on the measured capacitance and the measured temperature.

Among other advantages, the disclosed methods, devices and systems provide a reliable and cost-effective way to determine whether a spring is in a compressed state or an expanded state. In some embodiments, an exemplary advantage is that the disclosed methods, devices and systems may determine the state of the spring without having to make physical contact with the spring (or with any component that is in physical contact with the spring), as such physical contact may interfere with the operation of the spring. In some embodiments, another exemplary advantage is that the disclosed methods, devices, and systems may be mounted on or integrated with thin, flexible sheets that may be adhered to the external surface of existing medication-delivery devices, so as to provide such existing or previously-available devices with sensing and communication capabilities in a low-cost manner, and without having to make any modifications to the internal components of such delivery devices. In some embodiments, another exemplary advantage of the disclosed methods, devices, and systems is that by taking an ambient temperature (and/or a temperature of the sensing system or sensed compression spring) into account, the disclosed methods, devices, and systems can detect whether the compression spring is in a compressed state or expanded state with greater accuracy and reliability. Other advantages will be recognized by those of ordinary skill in the art.

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 is a logical block diagram depicting an exemplary sensing system for determining a state of a compression spring, wherein the depicted compression spring is in a compressed state, according to some embodiments.

FIG. 2 shows the sensing system when the compression spring is in an expanded state.

FIG. 3A depicts another embodiment of the sensing system that includes a capacitive shield, according to some embodiments.

FIG. 3B depicts another embodiment of the sensing system that includes plates comprising interleaved sets of fingers to form an interdigital capacitor, according to some embodiments.

FIG. 4A is an electrical circuit diagram that models the electrical circuit of the sensing system depicted in FIG. 1.

FIG. 4B is an electrical circuit diagram that models the electrical circuit of the sensing system depicted in FIG. 2.

FIG. 5A is an electrical circuit diagram that models the electrical circuit of the sensing system depicted in FIG. 3, when the compression spring is in its compressed state.

FIG. 5B is an electrical circuit diagram that models the electrical circuit of the sensing system depicted in FIG. 3, when the compression spring is in its expanded state.

FIG. 6 depicts an exemplary process for determining a state of a compression spring, according to some embodiments.

FIG. 7 illustrates an exemplary medication-delivery device in its initial pre-use configuration, according to some embodiments.

FIG. 8 illustrates the medication delivery device after its end cap has been removed, but before the device has been activated to deliver medication.

FIG. 9 illustrates the medication delivery device after the device has been activated to deliver medication.

FIG. 10 shows an exemplary label wrapped around an external surface of the medication delivery device, according to some embodiments.

FIG. 11 shows the label after it has been unwrapped and laid flat.

FIG. 12 shows a view of the back of the label.

FIG. 13 shows a second exemplary label wrapped around an external surface of the medication delivery device, according to some embodiments.

FIG. 14 shows a view of the back of the second label.

FIG. 15 shows a view of a back of a third exemplary label that uses plates comprising sets of interleaved fingers, according to some embodiments.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

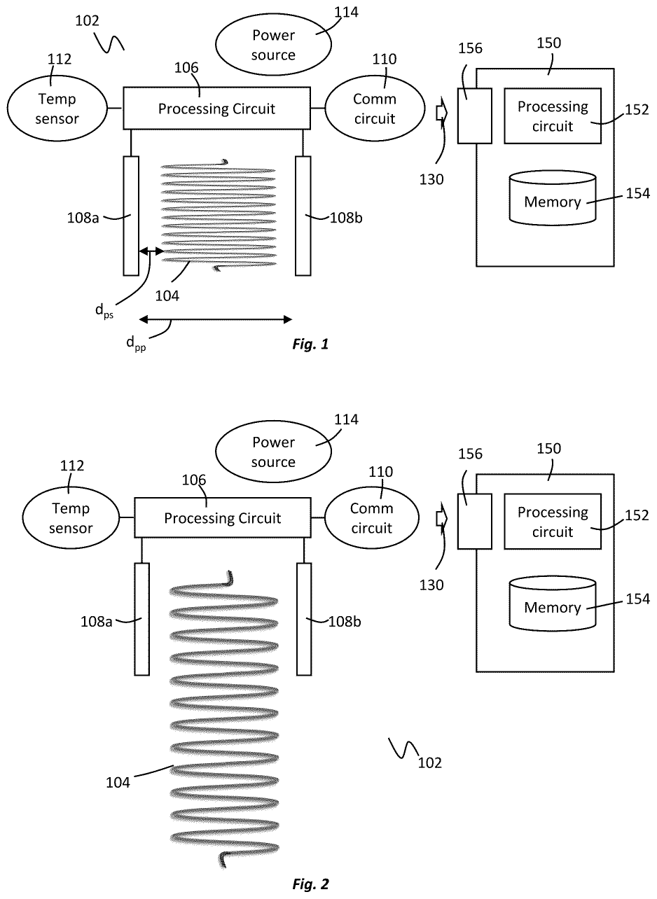

FIG. 1 is a logical block diagram depicting an exemplary sensing system 102 for determining a state of a compression spring 104, according to some embodiments. Compression spring 104 may be any suitable spring formed at least partially of an electrically conductive material (e.g., metal). The components of system 102 may receive power from a power source 114, which may be a battery, supercapacitor, a utility power grid, or a harvested power source that receives power inductively and wirelessly from an external source (e.g., from external device 150, described below). System 102 includes a first capacitive plate 108a configured to be positioned on a first side of the spring 104 and a second capacitive plate 108b configured to be positioned on a second side circumferentially spaced from the first side of the spring 104 such that the spring 104 is disposed between the first plate 108a and the second plate 108b (plates 108a and 108b are collectively referred to as plates 108). Plates 108 may take the form of any substantially planar or curved sheet formed at least partially of conductive metal (or another conductive material). System 102 further includes a temperature sensor 112 configured to sense a temperature. The sensed temperature may be a temperature of the spring 104, of one or both of plates 108, a temperature of any other component of system 102 described herein, a temperature of a device or system to which system 102 is mounted or integrated with, and/or an ambient temperature, according to different embodiments. Temperature sensor 112 may take the form of any suitable sensor for sensing temperature, such as but not limited to a thermistor (e.g., a negative temperature coefficient (NTC) thermistor or a resistance temperature detector (RTD)), a thermocouple, or a semiconductor-based temperature sensor.

Plates 108 and temperature sensor 112 are coupled to processing circuit 106. Processing circuit 106 may take the form of a processor (e.g., a microprocessor or microcontroller, field-programmable gate arrays (FPGAs) and/or digital signal processors (DSPs, or any combination of the foregoing) configured to execute logic stored in a memory (not shown) to perform the operations described herein. The term “logic”, “control logic”, “instructions” or “application” as used herein may include software and/or firmware executing on any of the aforementioned processing circuits. The memory may be any suitable computer readable medium that is accessible by processing circuit 106 and includes both volatile and non-volatile memory. Exemplary memory includes random-access memory (RAM), read-only memory (ROM), electrically erasable programmable ROM (EEPROM), flash memory, a magnetic storage device, optical disk storage, or any other suitable medium which is configured to store data and which is accessible by processor circuit 106, whether directly or indirectly via one or more intermediary devices or wired or wireless communication links. Although the preceding description assumes that the memory is separate from but communicably coupled to processing circuit 106, in some embodiments the memory may also be integrated with processing circuit 106. In some embodiments, instead of a processor that executes logic stored in memory, processing circuit 106 may take the form of hard-wired logic, e.g., a state machine and/or an application-specific integrated circuit (ASIC) that performs the functions described herein. In sensing system 102, processing circuit 106 is electrically coupled with plates 108 so as to measure an electrical capacitance between plates 108a and 108b. Similarly, processing circuit 106 is also communicably coupled to temperature sensor 112 to determine the temperature sensed by sensor 112 based on a signal output by sensor 112. Although processing circuit 106 and temperature sensor 112 are illustrated as separate elements in FIGS. 1-2, in some embodiments, temperature sensor 112 and processing circuit 106 may be integrated into a single integrated circuit.

Processing circuit 106 may also be communicably coupled with a communication circuit 110. Communication circuit 110 may take the form of circuitry configured to communicate data to and/or from an external device 150 using any suitable wireless transmission protocol, such as but not limited to a cellular transmission protocol, Bluetooth Low Energy (BLE), Near Field Communication (NFC), and/or Radio Frequency Identification (RFID). Processing circuit 106 may use communication circuit 110 to communicate to external device 150 at least one of the measured capacitance, the measured temperature, and data indicative of whether the compression spring is in the compressed state or the expanded state. Although processing circuit 106 and communication circuit 110 are illustrated as separate elements in FIGS. 1-2, in some embodiments, communication circuit 110 and processing circuit 106 may be integrated into a single integrated circuit (potentially along with temperature sensor 112).

External device 150 may comprise any device that receives, stores, and/or processes data from communication circuit 110 via a wireless signal 130 received by a communication circuit 156. Exemplary external devices include a smartphone, a smartwatch, a tablet, a laptop, a desktop PC, a wireless hub, and/or a WiFi access point. Wireless signal 130 may be an active signal, in which external device 150 receives signals transmitted by communication circuit 110, or it may be a passive signal, in which external device 150 senses modulations to a signal transmitted by communication circuit 156 caused by communication circuit 110 (e.g., a passive NFC signal). Similar to communication circuit 110, communication circuit 156 may comprise any circuitry configured to receive data from and/or transmit data to processing circuit 106 using any of the aforementioned wireless transmission protocols. External device 150 may further include a processing circuit 152, which may take the form of any of the aforementioned types of processing circuit, and a memory 154, which may also take the form of any of the aforementioned types of memory. In some embodiments, external device may further comprise a user interface for displaying data and/or receiving user input. For example, the user interface may comprise a graphical user interface (GUI) including a touchscreen display. The touchscreen display allows the user to interact with presented information, menus, buttons, and other data to provide information to the user or to receive user input from the user. Alternatively, or in addition, a keyboard, keypad, microphone, mouse pointer, or other suitable user input device may be provided. External device 150 may also comprise a separate communication circuit configured to communicate with other devices (e.g., using a long-range or cellular wireless transmission protocol).

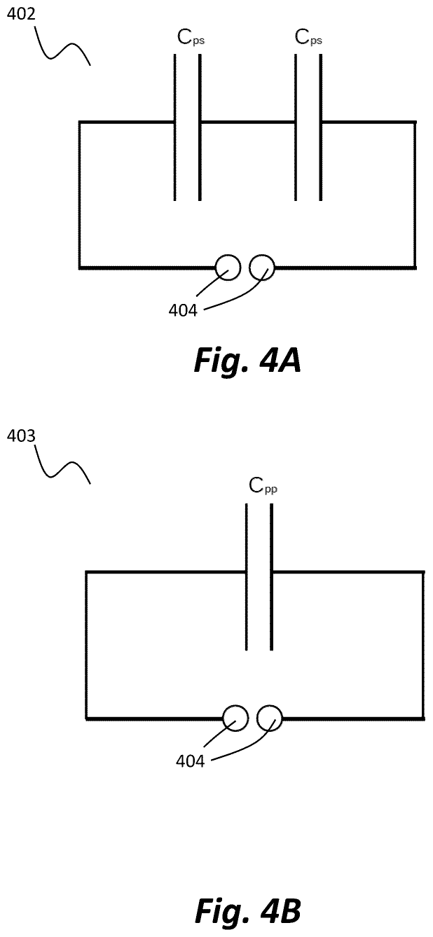

FIG. 1 shows the sensing system 102 when the compression spring 104 is in a compressed state. When in this compressed state, the coils of spring 104 are closely spaced such that the sides of the spring act as capacitive plates positioned between plate 108a and 108b. As such, when spring 104 is in its compressed state, the spring 104 and the plates 108 may be modeled using electrical circuit diagram 402 depicted in FIG. 4A. In electrical circuit diagram 402, the terminals 404 are considered to be connected to processing circuit 106. Cₚₛ represents the capacitance formed by one of the plates 108 and the sides of the compressed spring 104. Cₚₛ may be calculated according to Equation 1 below:

$${C}_{p s} = ε \cdot \frac{A}{{d}_{p s}} \qquad Eqn . 1$$

Where ε is the dielectricity of the material between one of the plates 108 and the sides of spring 104 (here, mostly air, but may also include an intervening layer of plastic or other material), A is the surface area of plate 108 (e.g., the product of length and width) and dps is the distance between one of the plates 108 and the side of spring 104 (see FIG. 1).

Since there are two plates 108, the total capacitance measured by processing circuit 106 between terminals 404 when spring 104 is in the compressed state (C_{total,comp}) is given by Equation 2 below:

$${C}_{total , comp} = \frac{{c}_{p s}}{2} \qquad Eqn . 2$$

This is because the relationship between the total capacitance (Cₜₒₜₐₗ) of two capacitors (C₁ and C₂) arranged in series is given by Equation 3 below:

$$\frac{1}{{c}_{t o t a l}} = \frac{1}{{c}_{1}} + \frac{1}{{c}_{2}} \qquad Eqn . 3$$

If C₁=C₂, as is the case here, then it follows that

$${C}_{total} = \frac{c}{2} .$$

FIG. 2 shows the sensing system 102 when the compression spring 104 is in an expanded state. When in this expanded state, the coils of spring 104 are spaced widely apart, or at least further apart than when the spring 104 is in its compressed state. When in this configuration, the sides of the spring no longer function as (or function less effectively as) capacitive plates positioned between plates 108a and 108b. As such, when spring 104 is in its expanded state, the spring 104 and the plates 108 may be modeled using electrical circuit diagram 403 in FIG. 4B. As with diagram 402, the terminals 404 in diagram 403 are considered to be connected to processing circuit 106. Cₚₚ represents the capacitance formed by the two plates 108, without any intervening capacitive plates positioned between them. Cₚₚ may be calculated according to Equation 4 below:

$${C}_{pp} = ε \cdot \frac{A}{{d}_{pp}} . \qquad Eqn . 4$$

Where ε is the dielectricity of the material between the plates 108 (again, mostly air), A is the surface area of the plates 108, and dₚₚ is the distance between the plates 108 (see FIG. 1).

dₚₛ and dₚₚ are configurable parameters depending on different embodiments. However, assuming for the sake of explication that dₚₚ=x·dps (where x is a configurable parameter) then the relationship between Cₚₚ and Cₚₛ is expressed by Equation 5 below:

$${C}_{pp} = \frac{{c}_{ps}}{x} \qquad Eqn . 5$$

Furthermore, since Cₚₚ is the only capacitance in electrical circuit diagram 403,
the total capacitance measured by processing circuit 106 between terminals 404 when spring 104 is in the expanded state (C_{total,exp}) is equal to Cₚₚ. Hence, the relationship between C_{total,exp} and C_{total,comp} is given by Equation 6 below:

$${C}_{total , exp} = \frac{2}{χ} \cdot {C}_{total , comp} \qquad Eqn . 6$$

Therefore, the capacitance measured by processing circuit 106 can be expected to change when spring 104 transitions between a compressed state and an expanded state (assuming x does not equal 2). For example, if x is equal to 3, then the capacitance observed by processing circuit 106 when the spring 104 is in its expanded state would be expected to be approximately ⅔ of the capacitance observed by processing circuit 106 when the spring 104 is in its compressed state. Hence, by measuring the capacitance of the system formed by spring 108 and spring 104, processing circuit 108 can determine whether the spring is in its expanded or compressed state.

The inventors have further appreciated that the capacitance measured by processing circuit 106 may vary depending on the temperature of the components of system 102, e.g., of spring 104, and plates 108. This may be because as the temperature increases, the gaps between links in spring 104 may increase or decrease due to thermal expansion. Furthermore, as the temperature increases, capacitive plates 108 may expand, thus increasing their surface area and consequently, the measured capacitance. Also, as the temperature increases, the distance between capacitive plates 108 and spring 104 may also vary. In addition, as the temperature increases, thermal noise and/or other temperature dependencies affecting measurements taken by the capacitance sensor on the processing circuit 106 may increase, thus increasing the measured capacitance. In some cases, the inventors have observed that the capacitance measured by processing circuit 106 may vary so much between a first (lower) temperature and a second (higher) temperature that it may become difficult to determine whether the spring 104 is in its compressed or expanded state based solely on the measured capacitance. In other words, in some use cases, the measured capacitance of the spring in its compressed state while at the first temperature may be too similar to the measured capacitance of the spring in its expanded state while at the second temperature. This makes it difficult to determine whether spring 104 is in its compressed or expanded state based solely on the measured capacitance, without taking the temperature of system 102 into account.

This is especially the case when system 102 is used to detect a state of a spring within a medication-delivery device that needs to be refrigerated for storage. Many types of injectable medication need to be stored at a lower temperature (e.g., between 36-46 degrees Fahrenheit, or 2 and 8 degrees Celsius) to prevent spoliation, but then need to be warmed up to a higher temperature (e.g., to room temperature, or between 65 and 75 degrees Fahrenheit) before being injected into the patient's body. Medication-delivery devices used to store and deliver such medications may come with instructions that instruct patients to store such devices in their fridge, but to take the device out and warm to room temperature before administration. In some embodiments, the measured capacitance of the spring in its compressed state while at the lower temperature (e.g., in the fridge) may be too similar to the measured capacitance of the spring in its expanded state while at the higher temperature (e.g., room temperature) to allow a sensing system 102 mounted on such a medication-delivery device to reliably determine which state the spring is in without taking temperature into account.

Accordingly, system 102 may include a temperature sensor 112 communicatively coupled to processing circuit 106. Sensor 112 allows processing circuit 106 to measure an ambient temperature, or a temperature of one or more components of system 102 (e.g., of spring 104 and/or plates 108). By taking the temperature measured by sensor 112 into account, processing circuit 106 may determine whether spring 102 is in the compressed state or the expanded state with greater accuracy.

For example, processing circuit 106 may determine that the spring 102 is in its compressed state when the measured capacitance of plates 108 is greater than a capacitance threshold. Conversely, processing circuit 106 may determine that spring 102 is in its expanded state when the measured capacitance is less than the capacitance threshold. Processing circuit 106 may be further configured to adjust the capacitance threshold based on the temperature measured by sensor 112. For example, the processing circuit 106 may increase the capacitance threshold as the temperature increases and decrease the capacitance threshold as the temperature decreases. The processing circuit 106 may do this in any of a number of ways, including: (i) defining two or more temperature bands (e.g., high/low, high/medium/low, or any other number of temperature bands) wherein each temperature band is delineated from the other temperature bands by temperature thresholds, and implementing logic that sets the capacitance threshold depending on which temperature band the current temperature falls within, and/or (ii) deriving the capacitance threshold to apply by multiplying the current temperature with a pre-programmed conversion constant. Any known mathematical or logical method for varying the capacitance threshold based on the temperature measured by sensor 112 may be used. In this way, processing circuit 106 may better determine whether spring 102 is in its compressed state or its expanded state based on both the measured capacitance of plates 108 and the temperature measured by sensor 112.

In some embodiments, processing circuit 106 may be configured to measure the capacitance between plates 108 and the temperature measured by sensor 112 and to communicate this measured data to processing circuit 152 in external device 150. Processing circuit 152, in turn, determines whether the spring 104 is in a compressed state or an expanded state based on the measured and received capacitance and temperature data. In yet other embodiments, processing circuit 106 and processing circuit 152 may work cooperatively to determine whether the spring is in a compressed state or an expanded state.

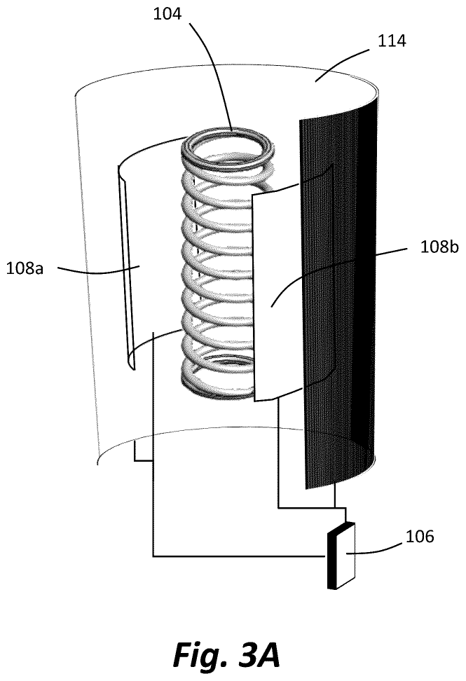

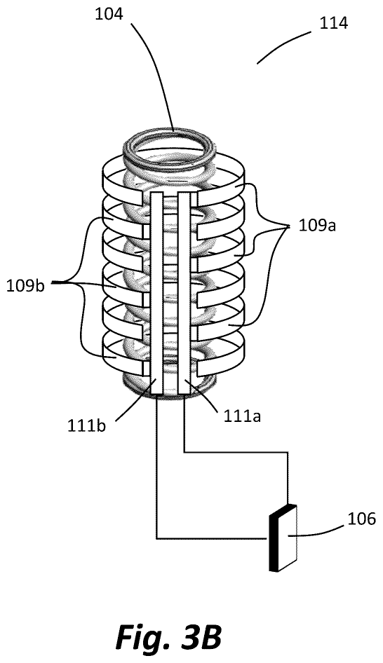

FIG. 3A depicts an embodiment of system 102 that includes a capacitive shield 114. For simplicity, temperature sensor 112 is not shown in FIG. 3A, although it should be understood that such a temperature sensor may be connected to and/or integrated with processing circuit 106. In this embodiment, plates 108a and 108b are configured as curved plates that conform to the curvature of spring 104. Furthermore, capacitive shield 114 may be configured as a metallic or conductive layer of material shaped like all or part of a cylinder, and which surrounds all or part of plates 108 and spring 104. Similar to plates 108, shield 114 may also be electrically connected to processing circuit 106. Shield 114 may decrease the effect of any conductive object (e.g., the hand of a user) touching or positioned close to plates 108 on the capacitance measured by processing circuit 106. By decreasing the magnitude of any such interference on the measured capacitance, shield 114 mitigates the possibility of such interference causing processing circuit 106 to mistakenly determine that the spring 104 is in its compressed state when it is in fact in its expanded state (or vice versa). In some embodiments, a polyester film (or a layer of some other dielectric material) (not shown) may be inserted between the plates 108 and shield 114 to separate these components from another.

FIG. 3B depicts another embodiment of system 102 that, instead of using two solid, curved plates 108a and 108b, uses two “plates” 109a, 109b (collectively, plates 109), wherein each plate comprises a set of spaced-apart parallel fingers formed at least partially of a metallic or conducting material that cover at least part of a surface of spring 104. The first “plate” comprising fingers 109a are electrically connected to a first conducting strip 111a, while the second “plate” comprising fingers 109b are electrically connected to a second conducting strip 111b. As depicted in FIG. 3B, the two plates are disposed such that the fingers of each plate are interleaved with one another in an alternating fashion, thus forming an interdigital capacitor. Conducting strip 111a and 111b are then communicably coupled to processing circuit 106. Although not depicted in FIG. 3B, it should be understood that a capacitive shield 114 may also be provided that surrounds all or part of the plates 109a, 109b and the conducting strips 111a, 111b.

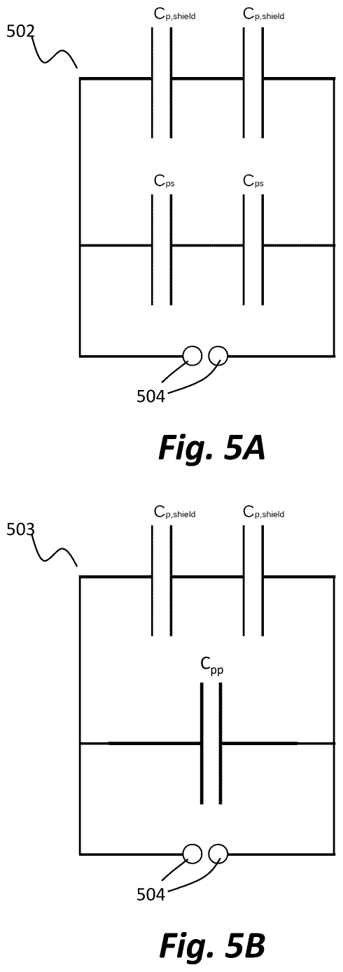

When spring 104 is in its compressed state, the spring 104, plates 108 (or 109), and shield 114 may be modeled using electrical circuit diagram 502 depicted in FIG. 5A. In electrical circuit diagram 502, the terminals 504 are considered to be connected to processing circuit 106. As before, Cₚₛ represents the capacitance formed by one of plates 108 (or 109) and the sides of the compressed spring 104. C_{p, shield} represents the capacitance formed by one of plates 108 or 109 and the shield 114. Cₚₛ may be calculated according to Equation 1. C_{p, shield} may be calculated using Equation 7 below:

$${C}_{p , shield} = {ε}_{PET} \cdot \frac{A}{{d}_{PET}} \qquad Eqn . 7$$

Where ε_{PET} is the dielectricity of the layer of dielectric material inserted between plates 108/109 and shield 114 (if the dielectric material is polyester, ε_{PET} may be expected to be approximately 2.8 times the dielectricity of air), A is the surface area of plates 108/109, and d_{PET} is the thickness of the layer of dielectric material. For example, d_{PET} in some embodiments may be approximately 40 μm.

Since the capacitance of two capacitors arranged in series is given by Equation 3, the capacitance (C_{offset}) of two capacitors with capacitance C_{p,shield} arranged in series is given by Equation 8 below:

$${C}_{offset} = \frac{{c}_{p , shield}}{2} \qquad Eqn . 8$$

Furthermore, since the capacitance of two capacitors arranged in parallel is given by the sum of the capacitance of both capacitors, the capacitance (C_{total,comp,shielded}) measured by processing circuit 106 when spring 104 is in its compressed state and shield 114 is present is given by Equation 9 below:

$${C}_{t o t a l , comp , shielded} = {C}_{total , comp} + {C}_{offset} \qquad Eqn . 9$$

When compression spring 104 is in its expanded state, the coils of spring 104 are spaced widely apart. As previously described, when in this configuration, the sides of the spring no longer function as (or function less effectively as) capacitive plates positioned between plates 108a and 108b (or 109a and 109b). As such, when spring 104 is in its expanded state, the spring 104, plates 108 or 109, and shield 114 may be modeled using electrical circuit diagram 503 in FIG. 5B. As with diagram 502, the terminals 504 are considered to be connected to processing circuit 106. As can be seen, diagram 503 is identical to diagram 403 except that two capacitors with capacitance C_{p, shield} are arranged in parallel with Cₚₚ. As such, the capacitance (C_{total, exp, shielded}) measured by processing circuit 106 when spring 104 is in its expanded state and shield 114 is present is given by Equation 10 below:

$${C}_{t o t a l , comp , shielded} = {C}_{total , comp} + {C}_{offset} \qquad Eqn . 10$$

Hence, the addition of shield 114 can be expected to add the capacitance C_{offset} to both C_{total,comp} when spring 104 is in its compressed state, and to C_{total,exp} when spring 104 is in its expanded state. By taking C_{offset} into account, the capacitance measured by processing circuit 106 can continue to serve as an indicator for whether spring 104 is in its compressed state or its expanded state.

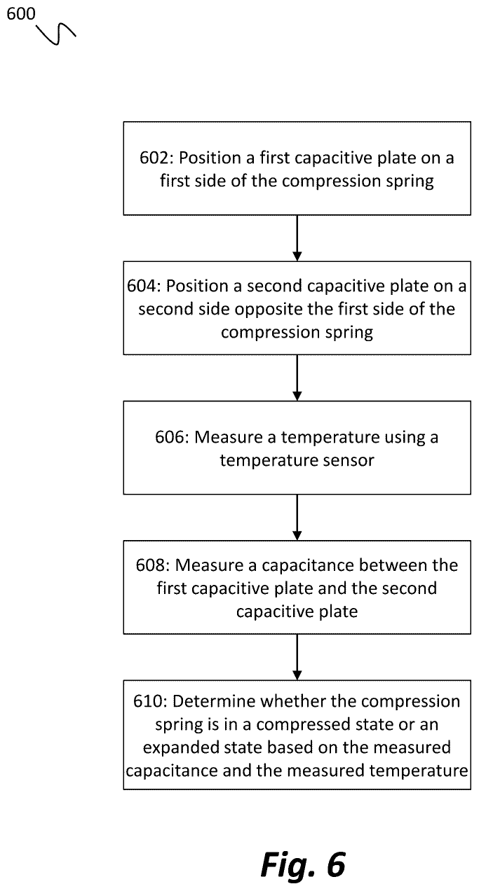

FIG. 6 depicts an exemplary process 600 for determining a state of a compression spring, according to some embodiments. Process 600 may be implemented using sensing system 102. Process 600 begins at step 602, in which a first capacitive plate (e.g., plate 108a or 109a) is positioned on a first side of the compression spring (e.g., spring 104). At step 602, a second capacitive plate (e.g., plate 108b or 109b) is positioned on a second side opposite the first side of the compression spring. In some embodiments, the second capacitive plate is positioned such that the compression spring is disposed between the first capacitive plate and the second capacitive plate. At step 606, a temperature is measured using a temperature sensor (e.g., temperature sensor 112). At step 608, a capacitance is measured between the first capacitive plate and the second capacitive plate. This measurement may be conducted using a processing circuit such as processing circuit 106. At step 610, a processing circuit determines whether the compression spring is in a compressed state or an expanded state based on the measured capacitance and the measured temperature. This determination may be done using any of the methods described herein. The processing circuit making this determination may be, for example, processing circuit 106. Alternatively, the processing circuit making this determination may be processing circuit 152 at external device 150, based on measured capacitance and temperature data measured and wirelessly communicated from processing circuit 106 via communications circuit 110 and 156. In some embodiments, this determination may be done by two or more processing circuits, e.g., processing circuit 106 and processing circuit 152, that are communicably coupled to each other (e.g., via a wired or wireless data link) and configured to cooperatively process the measured capacitance and/or temperature data to determine the state of spring 104.

In some embodiments, the compression spring may be part of a medication-delivery device, as described in further detail herein. The medication-delivery device may be configured to be activated to dispense medication and, when so activated, to transition the compression spring from a compressed state to an expanded state, or to transition the compression spring from the expanded state to the compressed state. The transition of the spring between states may be used to move a syringe assembly between a retracted position and an injection position or vice versa.

In some embodiments the method may further comprise reporting at least one of the measured capacitance, the measured temperature, and/or data indicative of whether the compression spring is in the compressed state or the expanded state to an external device via a wireless communication. This wireless communication may be done using communication circuit 110 and any of the wireless communication protocols described in connection therewith.

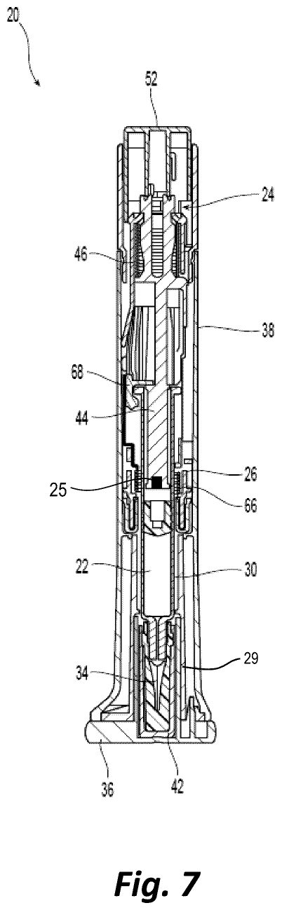

In FIGS. 7-9, a medication medication-delivery device 20 that may be used in conjunction with sensing system 102 is depicted in various operational states. One example of such a device and its operation is described in U.S. Pat. No. 8,734,394 B2 issued May 27, 2014 to Adams et al. and in U.S. Patent App. Pub. No. 2021/0093784 A1 published Apr. 1, 2021 to Adams et al., the entire disclosure of each of which is hereby incorporated herein by reference. Device 20 includes a syringe assembly 22, a drive mechanism 24, and a retraction mechanism 26. Syringe assembly 22 includes a barrel 30 forming a container body for holding a medication, and a piston 32 disposed within the barrel 30 for driving the medication outside the barrel. Syringe assembly 22 also includes a needle assembly 33 having a hollow injection needle 34 and a needle hub 35 which mounts needle 34 to syringe barrel 30. A lower body support member 29 coupled to device housing 38 surrounds needle 34. Advancing piston 32 within barrel 30 toward needle 34 dispenses medication through needle 34.

Devices described herein, such as device 20, may further comprise a medication, such as for example, within the syringe barrel 30. In another embodiment, a system may comprise one or more devices including device 20 and a medication. The term “medication” or “drug” refers to one or more therapeutic agents including but not limited to insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, combined GIP/GLP-1 agonists such as tirzepatide, oxyntomodulin analogs, oxyntomodulin derivatives, therapeutic antibodies including but not limited to IL-23 antibody analogs or derivatives, such as mirikizumab, IL-17 antibody analogs or derivatives, such as ixekizumab, therapeutic agents for pain-related treatments, such as galcanzeumab or lasmiditan, and any therapeutic agent that is capable of delivery by the devices described herein. The medication as used in the device may be formulated with one or more excipients. The device is operated in a manner generally as described above by a user, caregiver or healthcare professional to deliver medication to a patient. As used herein, the term “user” may refer to an operator of the devices described herein, and the term “patient” may refer to a person receiving the medication. In some cases, the user and the patient may be the same person (e.g., the patient is operating the devices described herein to give him/herself an injection). In other cases, the user and the patient may be different persons (e.g., the user may be a person providing care to the patient).

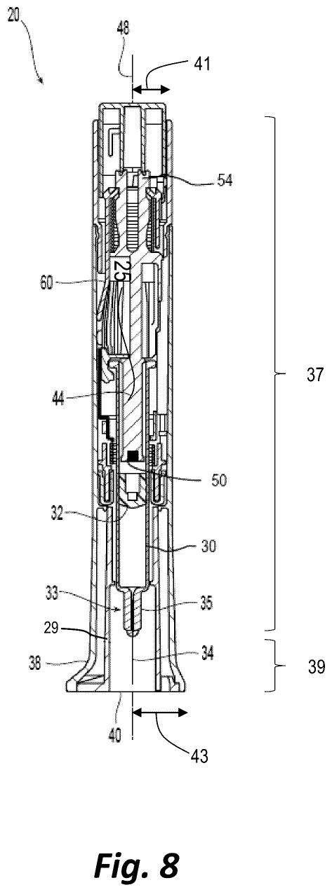

FIG. 7 illustrates device 20 in its initial, pre-use configuration. Here, an end cap 36 is secured to lower body support member 29 (which is in turn coupled to device housing 38). End cap 36 covers a proximal end opening 40 in housing 38. As used herein, distal and proximal refer to axial locations relative to an injection site when the apparatus is oriented for use at such site, whereby, for example, proximal end of the housing refers to the housing end that is closest to such injection site, and distal end of the housing refers to the housing end that is farthest from such injection site. Also as used herein, an “injection site” may refer to the exact spot on a patient's body that is injected by a needle, as well as body tissue surrounding the spot where the needle injects (e.g., within 1-5 cm or 1-10 cm of the spot where the needle punctures the patient's skin). Housing 38 may be formed from a plastic material and is shown extending generally longitudinally between a distal end in close proximity to an actuating button 52 and a proximal end in close proximity to the proximal end opening 40 along a longitudinal axis 48. As shown in FIG. 8, housing 38 may comprise a user-graspable portion 37 configured to be grasped by a hand of a user, the user-graspable portion 37 extending a radial distance 41 outward from longitudinal axis 48. In some embodiments, the radial distance 41 may be between 5-10 mm in length (e.g., in some embodiments, 5-8 mm may be a suitable length). Also as shown in FIG. 8, housing 38 may also comprise an outwardly-flared end portion 39 at a proximal end of the housing adjacent the proximal opening 40.

A needle guard 42 is mounted on syringe assembly 22 and covers and surrounds needle 34. End cap 36 and needle guard 42 protect the user from accidental needle pricks and also protect needle 34 from damage. When using device 20 to dispense medication, for example, injecting the medication into a patient, end cap 36 and needle guard 42 are first removed. FIG. 8 illustrates device 20 after removal of end cap 36 and needle guard 42 from syringe assembly 22, wherein the syringe assembly is in a storage position and device 20 is ready for a dispensing event.

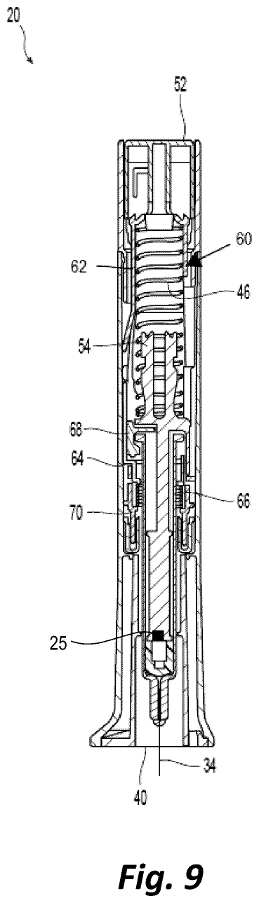

Syringe assembly 22 is moveable relative to the medication-delivery device 20 between a storage position and an injection position. FIG. 9 illustrates device 20 after the syringe assembly 22 has been moved relative to device 20 to an injection position from its storage position that is shown in FIG. 8. In the storage position (FIGS. 7 and 8), needle 34 is retracted to a position such that needle 34 is disposed within housing 38 of device 20. In the injection position (FIG. 9), needle 34 projects outwardly from housing 38 beyond proximal opening 40 in the proximal direction parallel to longitudinal axis 48 whereby needle 34 may be inserted into a patient.

Drive mechanism 24 includes a plunger 44 which engages piston 32. Drive mechanism 24 includes a spring 46 that drives plunger 44 in a translational movement. In the illustrated embodiment, spring 46 advances plunger 44 along a linear path defined by the longitudinal axis 48 of device 20. As plunger 44 is advanced, foot 50 of plunger 44 contacts piston 32. As the plunger 44 is further advanced, syringe assembly 22 is advanced along axis 48 from its storage position to its injection position. After advancement of syringe assembly 22 to its injection position, the continued proximal advancement of plunger 44 advances piston 32 proximally within barrel 30 from its initial piston position (shown in FIGS. 7 and 8) to its final piston position (shown FIG. 9) to cause medication to be dispensed from needle 34 in a dispensing event. Prior to any dispensing of medication and when syringe barrel 30 holds the full original volume of medication, piston 32 will be in its initial piston position. After advancing piston 32 the full extent of its travel length toward needle assembly 33, piston 32 will be in its final piston position proximate needle assembly 33 and the medication from within barrel 30 will have been discharged. In some embodiments, syringe assembly 22 will hold a single dose of medication which will be delivered in a single injection event and piston 32 will be advanced from its initial piston position to its final piston position in that single injection event to thereby deliver the entire single dose contents of syringe assembly 22. While the device is shown as a single use device, device 20 may also be configured as a multiple-use device with appropriate modifications.

The advancement of plunger 44 will generally not result in the dispensing of medication from syringe assembly 22 until after syringe assembly 22 has been advanced to the injection position. There are factors that may inhibit the medication from being dispensed before the syringe is advanced to the injection position. A factor may be the friction between piston 32 and barrel 30. Typically, piston 32 will be formed out of a rubber material and barrel 30 will be glass. The frictional resistance between these two components may be sufficient to prevent the advancement of piston 32 within barrel 30 until syringe assembly 22 is advanced to its injection position and engagement with a suitable stop member prevents the further advancement of syringe assembly 22. Additionally, the medication within the syringe may be somewhat viscous and thereby somewhat resistant to flowing out of needle 34. If necessary, modification of piston 32 and syringe barrel 30 to alter the frictional resistance of the dispensing motion of the engagement member 32 relative to syringe barrel 30 may limit or prevent the premature dispensing of medication before container 22 reaches its injection position.

To activate drive mechanism 24, a person depresses actuating button 52 at the distal end of device 20. Depressing button 52 disengages one or two elongate prongs 54 on plunger 44 from a shuttle assembly 60 thereby allowing spring 46 to expand so as to axially advance plunger 44. Spring 46 has a helical shape and surrounds prongs 54. The proximal end of spring 46 biasingly engages a flange on plunger 44.

Shuttle assembly 60 may include an upper shuttle member 62 and a lower shuttle member 64. Shuttle members 62, 64 are fixed together in the final assembly. In the final assembly, upper shuttle member 62 captures button 52 and spring 46 limiting the axial movement of these parts in the distal direction. Prongs 54 engage surfaces on upper shuttle 62 when the device is in the condition shown in FIGS. 7 and 8. Depressing button 52 causes tabs on button 52 to engage ramps (not shown) on prongs 54 to bias prongs 54 inwardly to disengage prongs 54 from upper shuttle member 62. After prongs 54 have been disengaged, spring 46 exerts a biasing force on a flange on plunger 44 to advance plunger 44 from the position shown in FIG. 8 to the position shown in FIG. 9 as spring 46 transitions from its compressed state to an expanded state. As plunger 44 is advanced, it moves syringe assembly 22 to the injection position and then advances piston 32 to dispense medication as discussed above.

After the dispensing event is complete, retraction mechanism 26 optionally moves syringe assembly 22 from the injection position shown in FIG. 9 back to a retracted position. More specifically, the retraction mechanism is adapted to move the medication container from the injection position to the retracted position in a retraction movement. The retracted position may be similar to the storage position in that the syringe assembly is drawn back into the housing 38 such that needle 34 no longer projects proximally from proximal opening 40 and is disposed entirely within housing 38. In some embodiments, the retracted position may be the same as the storage position. In other embodiments, however, a syringe assembly 22 in the retracted position may be located slightly proximal or distal to a syringe assembly in the storage position. In the illustrated embodiment, the retraction mechanism includes a spring 66, a syringe carrier and a rotary member 70 that acts as a follower. In yet other embodiments, the device 20 may include no retraction mechanism 26 such that the syringe assembly remains in its injection position indefinitely after the medication has been dispensed, until the syringe assembly is manually removed or repositioned by a user.

Plunger 44 may include an outrigger (not shown) which unlocks rotary member 70 as plunger 44 nears the end of its travel in the proximal direction. Rotary member 70 is rotationally secured to lower shuttle member 64 by engagement between a latch and a latching recess in lower shuttle member 64. The outrigger unlocks member 70 by depressing the latch. Spring 66 is torsionally preloaded and has one end engaged with member 70 and an opposite end engaged with shuttle assembly 60. Upon depression of the latch, spring 66 causes member 70 to rotate.

Member 70 is rotatable within housing 38 but is not axially moveable relative to housing 38. Other embodiments may include a member 70 that is also axially movable. The rotation of member 70 serves as a delay mechanism to prevent retraction mechanism 26 from retracting syringe assembly 22 until after the syringe assembly has finished delivering its dose of medication. The speed of rotation of member 70 may be adjusted by adjusting a viscosity of grease disposed on or around surfaces of member 70 that are in contact with housing 38—a more viscous grease results in slower rotation, while a less viscous grease results in faster rotation. A radial flange on rotary member 70 may engage a ledge within housing member 38 to limit the proximal movement of member 70. Spring 66 may also be compressively preloaded such that it is initially in a compressed state. In this compressed state, spring 66 may exert an axial force, torsional force, or both forces on member 70 to bias member 70 proximally to thereby maintain member 70 in an axial position where the radial flange of member 70 engages the interior ledge of housing member 38.

Shuttle assembly 60 may include axially extending channels or ribs that engage corresponding features on housing member 38 that allow shuttle assembly 60 to move axially within housing 38 but which prevent the relative rotation of shuttle assembly 60 relative to housing member 38. Shuttle assembly 60 is biased in the distal direction by spring 66 but is prevented from moving distally by engagement of a latch (not shown) before activation of drive mechanism 24. When rotary member 70 completes its rotation, it disengages the aforementioned latch, thus allowing shuttle assembly 60 to move distally under the biasing force of spring 66.

As shuttle assembly 60 moves distally, it carries syringe assembly 22 distally and moves it back to the retracted position. Also as shuttle assembly 60 moves, spring 66 transitions from its compressed state to an expanded state. Spring 66 biases the retraction mechanism 26 distally and thereby maintains syringe assembly 22 in its retracted position after an injection event. In some embodiments, as shuttle assembly 60 moves distally, spring 46 of drive mechanism 24 may also transition from an expanded state to a compressed state. A locking mechanism such as a detent on the shuttle assembly 60 and a recess on the housing 38 member may additionally provide a locking engagement to secure syringe assembly 22 in the retracted position with needle 34 disposed within housing 38 after an injection event whereby the user may then dispose or otherwise handle device 20 in a safe manner.

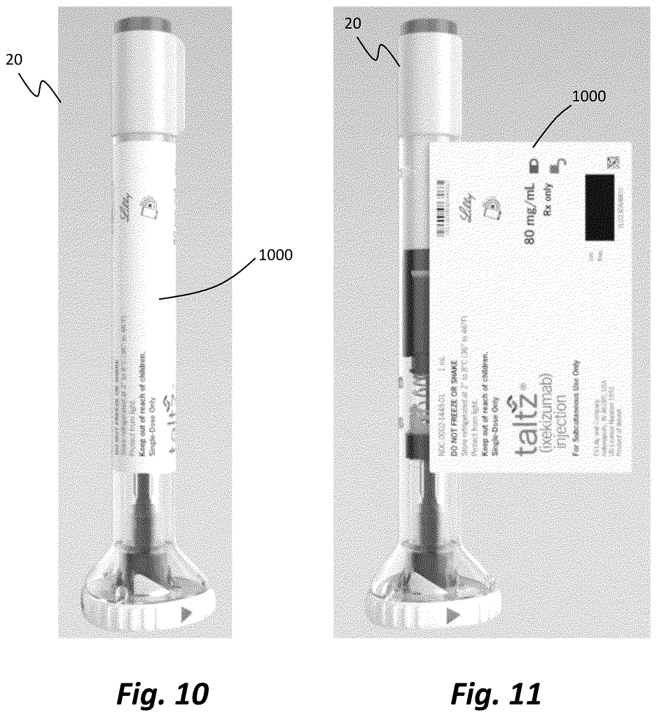

FIG. 10 shows a label 1000 wrapped around an external surface of user-graspable portion 37 of medication-delivery device 20, while FIG. 11 shows the label 1000 unwrapped and laid flat, according to some embodiments. The label 1000 may comprise a flexible paper and/or plastic material that may be attached to the exterior surface of device 20. Label 1000 may be attached using any of a variety of methods, including (but not limited to) using adhesive, adhesion film (e.g., polyurethane films), magnetic attachments, clip-on attachments, ultrasonic bonding/welding, injection molding/in-mold labeling, laser joining/welding, and the like. Label 20 may be printed with information regarding the device 20 and/or regarding the medication stored within device 20, such as the medication's name, manufacturer name, manufacturing batch/lot number, expiration date, instructions for use and/or storage, the amount of medication stored therein, and the like.

A sensing system 102 may be mounted on and/or integrated with label 1000 to provide label 1000 the ability to sense whether a compression spring within medication-delivery device 20 (e.g., spring 46 of drive mechanism 24 and/or spring 66 of retraction mechanism 26) is in a compressed state or an expanded state. This determination regarding the state of the compression spring within device 20 may indicate whether device 20 has been activated to deliver the medication, and/or whether device 20 has completed delivery of the medication. For instance, since spring 46 transitions from a compressed state to an expanded state when medication-delivery device 20 is activated to dispense the medication, a sensing system 102 mounted on and/or integrated with label 1000 may be used to detect the state of spring 46 and infer therefrom whether medication-delivery device 20 has been activated or not. As another example, since spring 66 transitions from a compressed state to an expanded state when medication-delivery device 20 retracts syringe assembly 22 at the end of an injection, a sensing system 102 mounted on and/or integrated with label 1000 may be used to detect the state of spring 66 and infer therefrom whether medication-delivery device 20 has completed its retraction of syringe assembly 22, and/or completed its injection or not. As yet another example, since, in some embodiments spring 46 may transition from an expanded state to a compressed state when medication-delivery device 20 retracts syringe assembly 22 at the end of an injection, a sensing system 102 mounted on and/or integrated with label 1000 may be used to detect the state of spring 46 and infer therefrom whether medication-delivery 20 has completed its retraction of syringe assembly 22, and/or completed its injection or not. The sensing system 102 mounted on and/or integrated with label 1000 may also communicate its determination regarding the state of the sensed compression spring to an external device, such as external device 150 using communication circuit 110. The external device may infer from this information whether the user has taken and/or completed an injection, log such information and/or inference, and/or transmit such information and/or inference to other devices for storage, analysis, and/or further action.

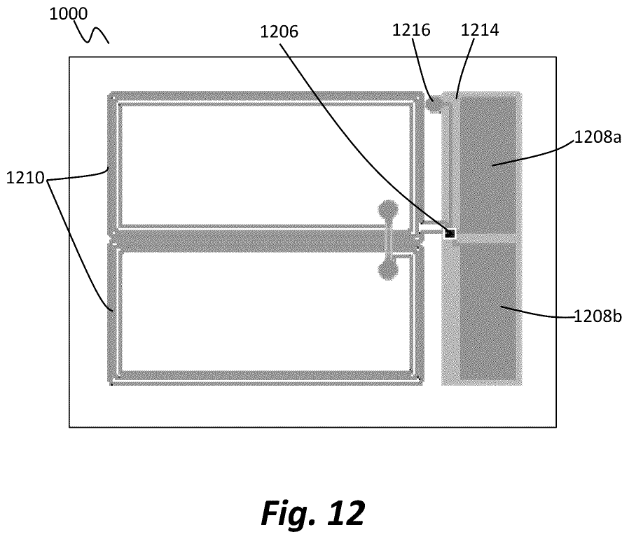

FIG. 12 shows a view of a back surface of the exemplary label 1000 (e.g., the surface configured to wrap around and/or adhere to exterior surface of medication-delivery device 20) that is attached to and/or integrated with a sensing system 102, according to a first embodiment. Label 1000 comprises a processing circuit 1206 that is configured similarly to, and performs the same functions as, processing circuit 106. Although not shown separately, processing circuit 1206 may also comprise a temperature sensor similar to sensor 112, as previously described. In some embodiments, processing circuit 1206 may be a package-less “bumped die” integrated circuit formed with an on-board temperature sensor and a capacitive sensing interface-such a circuit may have an exemplary thickness of 0.15 mm or less and be mounted on a flexible substrate, thus allowing the circuit to be easily adhered to the barrel-shaped user-graspable portion 37 of medication-delivery device 20.

Label 1000 further comprises capacitive plates 1208a and 1208b (collectively referred to herein as plates 1208), which are configured similarly to, and perform the same functions as, plates 108a and 108b. Label 1000 further comprises an electrically conductive shield 1214 that covers capacitive plates 1208 and shields capacitive plates 1208 from contact with (or capacitive interference from) a user's hand when label 1000 is wrapped around the exterior surface of the user-graspable portion 37 of medication-delivery device 20. Shield 1214 is separated from capacitive plates by a thin layer of dielectric material, such as a thin film of polyester, as previously described. Shield 1214 may also be electrically connected to processing circuit 1206 via a wire or conductive trace that passes through this thin layer of dielectric material via a through-hole 1216. Label 1000 further comprises Near Field Communication (NFC) loop antennas 1210 that are communicably coupled with processing circuit 1206—these NFC antennas correspond to communication circuit 110 as previously described. In addition to communicating data to an external device, NFC antennas 1210 may also be used to collect power inductively and wirelessly from said external device.

The back surface of label 1000 may be mounted on and/or attached to the exterior surface of medication-delivery device 20 (or some other surface of device 20). For example, label 1000 may be wrapped around and adhesively secured to the outer surface of the user-graspable portion 37 of medication-delivery device 20. When label 1000 is so attached, plates 1208 may be positioned on opposite sides of spring 46, spring 66, or some other spring within medication-delivery device 20 and may be used to detect whether the sensed spring is in a compressed state or an expanded state, using any of the techniques described herein. Data regarding the state of the sensed spring may be used by processing circuit 1206 to determine whether device 20 has been activated to dispense medication, and/or has finished dispensing medication, as previously described. Alternatively, or in addition, label 1000 may be configured to wirelessly communicate data regarding the capacitance between plates 1208 measured by processing circuit 1206, and/or the temperature sensed by its onboard temperature sensor, to a processing circuit on an external device (e.g., processing circuit 152 in external device 150), thus allowing the external device to determine whether the sensed spring is in a compressed state or an expanded state.

In some embodiments, label 1200 may be modified to remove shield 1214. As previously discussed, shield 1214 may function to decrease capacitive interference from a user's hand when the user is gripping the user-graspable portion 37 of medication-delivery device 20. However, in some embodiments, shield 1214 may be intentionally removed such that capacitive plates 1208 may be used to sense whether a user is grasping medication-delivery device 20 or not. When a shield 1214 is not installed, the capacitance of plates 1208 measured by processing circuit 1206 may be expected to change depending on whether a user is gripping the user-graspable portion 37 of medication-delivery device 20. That change in capacitance may be used to allow processing circuit 1206 to determine whether a user is grasping the device 20 or not.

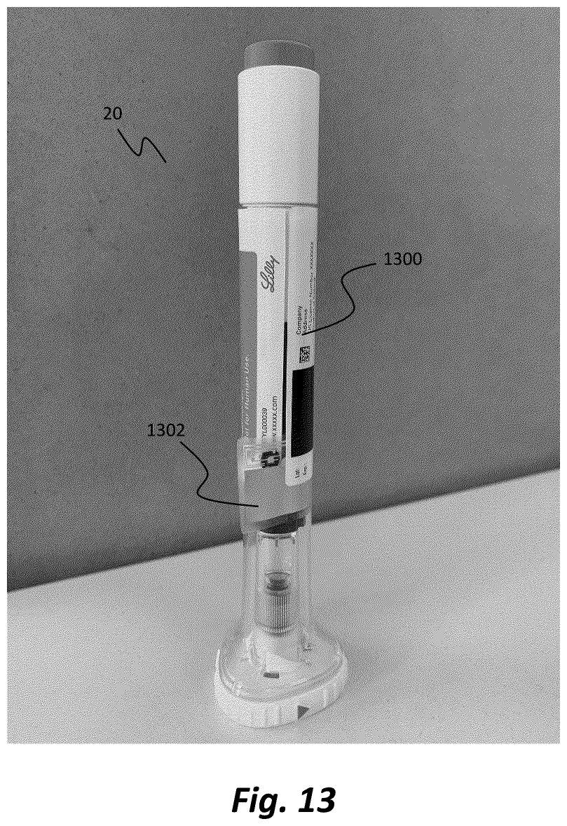

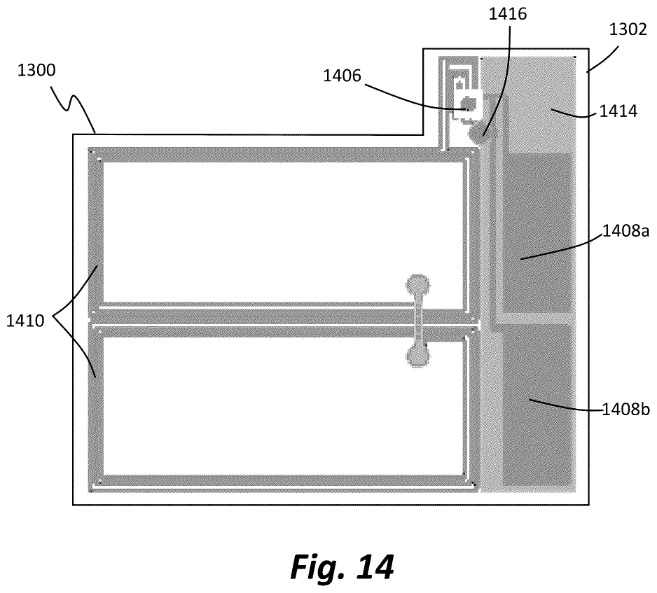

FIG. 13 shows another exemplary label 1300 wrapped around an external surface of user-graspable portion 37 of medication-delivery device 20, according to some embodiments. FIG. 14 shows a view of the back surface of label 1300. Label 1300 is configured similarly to label 1000, except that label 1300 includes an additional flag 1302 that extends from one side of the otherwise rectangular label 1300. When label 1300 is wrapped onto medication-delivery device 20, flag 1302 is configured to overlap a portion of label 1300, as shown in FIG. 13.

Similar to label 1000, label 1300 also comprises a processing circuit 1406 that is configured similarly to, and performs the same functions as, processing circuit 106. Unlike processing circuit 1306, processing circuit 1406 need not be adhered directly to the surface of the medication-delivery device 20, and therefore does not need to be as thin and/or flexible as processing circuit 1306. For example, processing circuit 1406 may be formed from a packaged NFC chip having a thickness of approximately 1 mm or more. The packaged NFC chip may be equipped with an on-board temperature sensor and a capacitive sensing interface and need not be mounted on a flexible substrate that enables the chip to bend. Such packaged chips may be easier to purchase and/or manufacture than the thinner and more flexible processing circuit 1306, thus decreasing manufacturing costs.

In other respects, label 1300 is substantively similar to label 1000. Label 1300 also comprises capacitive plates 1408a and 1408b, which is configured similarly to, and perform similar functions as, capacitive plates 1208 and 108. Label 1300 further comprises a conductive shield 1414 separated from capacitive plates 1408 by a thin insulator film (e.g., a polyester film). Shield 1414 is configured similarly to, and performs similar functions as, shields 1214 and 114, and is electrically connected to processing circuit 1406 via a wire or conductive trace that passes through the polyester film via through-hole 1416. Label 1300 also includes NFC loop antennas 1410 that are communicably coupled with processing circuit 1306—these NFC antennas correspond to communication circuit 110 as previously described. In addition to communicating data to an external device, NFC antennas 1410 may also be used to harvest power inductively and wirelessly from said external device.

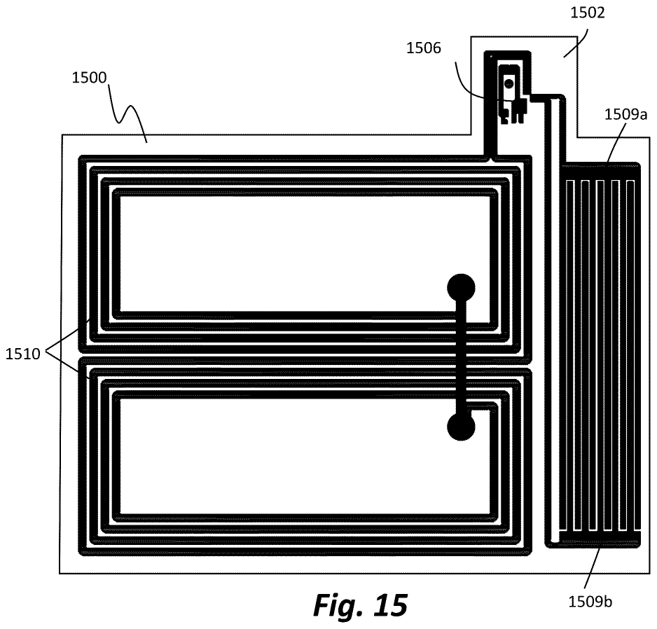

FIG. 15 shows a view of the back surface of yet another exemplary label 1500. Label 1500 is configured similarly to label 1300, except that instead of using capacitive plates 1408a and 1408b, label 1500 uses a first plate 1509a comprising a set of fingers that are interleaved with a second set of fingers from a second plate 1509b to form an interdigital capacitor. In other respects, label 1500 is substantively similar to label 1300. Label 1500 also comprises a processing circuit 1506 that is configured similarly to processing circuit 106, as well as NFC loop antennas 1510 that are communicably coupled with processing circuit 1506—these NFC antennas correspond to communication circuit 110 as previously described. Although not depicted in FIG. 15, it should be understood that label 1500 may also be provided with a capacitive shield similar to shields 1414, 1214, and/or 114.

While the previous embodiments have been described as systems for detecting whether a spring is in a compressed state or an expanded state, in some embodiments, the processing circuit (e.g., processing circuit 106, 152, 1206, 1406, and/or 1506) may be configured to detect whether a body part of a user is positioned close to the capacitive plates. The processing circuit may perform this function either in addition to, or as an alternative to, the aforementioned methods for detecting the compressed/expanded state of the spring.

For example, in embodiments where system 102 is used in a drug-delivery device (e.g., device 20 described above in FIGS. 7-9), the processing circuit may use the measured capacitance of the capacitive plates (e.g., plates 108, 1208, 1408, and/or 1508) to determine whether a user's hand is gripping the drug-delivery device (e.g., gripping the user-graspable portion 37 shown in FIG. 8). In such embodiments, the capacitive shield (e.g., shield 114, 1214, 1414) may be omitted. When the user grips the drug-delivery device, the processing circuit may detect a measurable increase in the measured capacitance of the capacitive plates. Based on this detected increase in capacitance, processing circuit 106 and/or processing circuit 152 may determine that the drug-delivery device either is being gripped by a hand of a user or is not being gripped by a hand of the user.

In some embodiments, a single set of capacitive plates 108 (e.g., capacitive plates 108a and 108b) may be used to detect both whether a user is gripping a drug-delivery device as well as whether the spring is in a compressed state or an expanded state. For example, when the processing circuit measures a capacitance greater than a first maximum capacitance threshold, the processing circuit may determine that the spring is in a compressed state and the user's hand is gripping the drug-delivery device. When the processing circuit measures a capacitance lower than a second minimum capacitance threshold, the processing circuit may determine that the spring is in an expanded state and the user's hand is not gripping the drug-delivery device. When the processing circuit measures a capacitance that is between the first maximum capacitance threshold and the second minimum capacitance threshold, the processing circuit may determine that either (1) the spring is in an expanded state and the user's hand is gripping the drug-delivery device or (2) the spring is in a compressed state and the user's hand is not gripping the drug-delivery device.

In some embodiments, a drug-delivery device (or a label for a drug-delivery device) may be provided with multiple sets of capacitive plates. A first set of plates may be positioned and configured to detect whether spring 104 is in a compressed state or an expanded state. This first set of plates may be provided with a capacitive shield similar to shields 114, 1214, and 1414 described previously. A second set of capacitive plates (or an additional single capacitive plate) may be positioned and configured to determine whether a user's hand is gripping the drug-delivery device. This second set of plates may not be shielded. In this way, the processing circuit 106 may determine both (1) whether the spring is in an expanded state or a compressed state based on the capacitance measured by the first set of plates and (2) whether the user is gripping the drug-delivery device based on the capacitance measured by the second set of plates.

In yet other embodiments, a drug-delivery device (or a label for a drug-delivery device) may be provided with three sets of capacitive plates. A first set of plates may be positioned and configured to determine whether spring 46 is in a compressed state or an expanded state. This determination may help processing circuit 106 determine whether the drug-delivery device has initiated an injection, since spring 46 expands when an injection is initiated. A second set of plates may be positioned and configured to determine whether spring 66 is in a compressed state or an expanded state. This determination may help processing circuit 106 determine whether the drug-delivery device has completed an injection, since spring 66 expands when an injection is completed, and the syringe assembly is retracted. A third set of plates (or an additional single plate) may be positioned along user-graspable portion 37 of drug-delivery device 20 and configured to determine whether a user is gripping the drug-delivery device. The first and second set of plates may be provided with capacitive shielding, while the third set of plates may be unshielded.

The capacitive plates and processing circuits in the aforementioned embodiments may also be modified to enable the processing circuit to determine whether the device is being gripped by only one hand of the user or being gripped by two hands. For instance, the capacitive plates in the aforementioned may be enlarged (or multiple sets of capacitive plates may be provided) to cover a relatively large area of the user-graspable portion 37 of the drug-delivery device 20. If the device is being gripped by two hands, the capacitance of the capacitive plates measured by the processing circuit may be higher than if the device is being gripped by one hand only. In this way, the processing circuit may determine whether the device is being gripped by one hand, by two hands, or not being gripped at all. If the aforementioned capacitive plates are also placed close to the end of the drug-delivery device that comes into contact with the user's injection site during an injection, the aforementioned capacitive plates may also be used to detect whether the drug-delivery device is in contact with an injection site on the user's body. This additional detection functionality for detecting contact with an injection site may be in addition to, or as an alternative to, the aforementioned detection functionality, i.e., detecting a compression and/or expansion state of the spring and detecting whether the delivery device is being gripped by one or more hands of the user.

The terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

Various aspects are described in this disclosure, which include, but are not limited to, the following aspects:

1. A medication delivery device with a sensing system for determining a state of a compression spring of the medication delivery device, the sensing system comprising: a first capacitive plate and a second capacitive plate configured such that the compression spring is disposed between at least a portion of the first capacitive plate and at least a portion of the second capacitive plate; a temperature sensor; and at least one processing circuit connected to the first capacitive plate, the second capacitive plate, and the temperature sensor, the at least one processing circuit configured to: measure a capacitance between the first capacitive plate and the second capacitive plate, measure a temperature based on a signal output by the temperature sensor, and determine whether the compression spring is in a compressed state or an expanded state based on the measured capacitance and the measured temperature.

2. The medication delivery device of claim 1 wherein the at least one processing circuit comprises a first processing circuit and a second processing circuit, and wherein: the first processing circuit is configured to measure the capacitance, measure the temperature, and communicate the measured capacitance and the measured temperature to the second processing circuit; and the second processing circuit is configured to determine, based on the communicated capacitance and the communicated temperature, whether the compression spring is in the compressed state or the expanded state.

3. The medication delivery device of claim 1, wherein the at least one processing circuit consists of a single processing circuit.

4. The medication delivery device of any of claims 1-3, wherein the temperature sensor and the at least one processing circuit are integrated into a single integrated circuit.

5. The medication delivery device of any of claims 1-4, wherein the sensing system is configured to receive harvested power wirelessly from an external power source.

6. The medication delivery device of any of claims 1-5, wherein the at least one processing circuit is configured to determine that the compression spring is in the compressed state when the measured capacitance is greater than a capacitance threshold, and to determine that the compression spring is in the expanded state when the measured capacitance is less than the capacitance threshold.

7. The medication delivery device of claim 6, wherein the at least one processing circuit is configured to adjust the capacitance threshold based on the measured temperature.

8. The medication delivery device of any of claims 1-7, wherein: the sensing system further comprises a wireless communication interface; and the at least one processing circuit is connected with the wireless communication interface and is configured to communicate to an external device via the wireless communication interface at least one of the measured capacitance, the measured temperature, and data indicative of whether the compression spring is in the compressed state or the expanded state.

9. The medication delivery device of any of claims 1-8, wherein the first capacitive plate comprises a first plurality of fingers, the second capacitive plate comprises a second plurality of fingers, and the first capacitive plate and the second capacitive plate are disposed such that the first plurality of fingers is interleaved with the second plurality of fingers.

10. The medication delivery device of any of claims 1-9, wherein the at least one processing circuit is configured to determine whether a hand of a user is gripping the medication delivery device based on the measured capacitance.

11. The medication delivery device of any of claims 1-9, further comprising at least a third capacitive plate disposed on a user-graspable portion of the medication delivery device, wherein the at least one processing circuit is configured to determine whether a hand of a user is gripping the medication delivery device based on a capacitance measured by the third capacitive plate.

12. The medication delivery device of any of claims 1-11, further comprising a medication.

13. An injection system comprising: a medication-delivery device housing; a syringe assembly at least partially disposed within the medication-delivery device housing, the syringe assembly including a reservoir configured to hold a medication and an injection needle; a drive mechanism at least partially disposed within the medication-delivery device housing configured to, upon activation by a user, dispense medication from the syringe assembly via the injection needle, the drive mechanism including a compression spring that is configured to transition from a compressed state to an expanded state, or to transition from an expanded state to a compressed state, after said activation; and a sensing system comprising: a first capacitive plate and a second capacitive plate configured such that the compression spring is disposed between at least a portion of the first capacitive plate and at least a portion of the second capacitive plate; a temperature sensor, and at least one processing circuit configured to: measure a capacitance between the first capacitive plate and the second capacitive plate, and measure a temperature based on a signal output by the temperature sensor.

14. The injection system of claim 13, wherein the temperature sensor and the at least one processing circuit are integrated into a single integrated circuit.

15. The injection system of any of claims 13-14, further comprising a wireless communication interface, wherein the at least one processing circuit is configured to communicate to an external device, via the wireless communication interface, the measured capacitance and the measured temperature so as to allow the external device to determine whether the compression spring is in a compressed state or an expanded state based on the measured capacitance and the measured temperature.

16. The injection system of any of claims 13-14, wherein the at least one processing circuit is further configured to determine whether the compression spring is in a compressed state or an expanded state based on the measured capacitance and the measured temperature.

17. The injection system of claim 16, further comprising a wireless communication interface, wherein the at least one processing circuit is configured to communicate to an external device, via the wireless communication interface, data indicative of whether the compression spring is in the compressed state or the expanded state.

18. The injection system of any one of claims 15 and 17, wherein the wireless communication interface comprises a Near Field Communication (NFC) circuit.

19. The injection system of any of claims 13-18, wherein the first capacitive plate, the second capacitive plate, the temperature sensor, and the at least one processing circuit are attached to a label secured to an external surface of the medication-delivery device housing.

20. The injection system of any of claims 13-19, wherein: the medication-delivery device housing defines an injection aperture; the syringe assembly is movable between a storage position in which the injection needle does not extend through the injection aperture and an injection position in which the injection needle does extend through the injection aperture; and the compression spring is configured to, after the medication-delivery device is activated, transition from the compressed state to the expanded state so as to move the syringe assembly from the injection position to the storage position after the medication has been dispensed.

21. The injection system of any of claims 13-19, wherein the compression spring is configured to transition from the compressed state to the expanded state so as to dispense medication from the reservoir through the injection needle.

22. The injection system of any of claims 13-21, further comprising a metallic shield that covers the first capacitive plate and the second capacitive plate.

23. The injection system of any of claims 13-Error! Reference source not found, wherein the first capacitive plate comprises a first plurality of fingers, the second capacitive plate comprises a second plurality of fingers, and the first capacitive plate and the second capacitive plate are disposed such that the first plurality of fingers is interleaved with the second plurality of fingers.

24. The injection system of any of claims 13-23, wherein the at least one processing circuit is configured to determine whether a hand of a user is gripping the medication delivery device based on the measured capacitance.

25. The injection system of any of claims 13-23, further comprising at least a third capacitive plate disposed on a user-graspable portion of the medication delivery device, wherein the at least one processing circuit is configured to determine whether a hand of a user is gripping the medication delivery device based on a capacitance measured by the third capacitive plate.

26. The injection system of any of claims 13-25, further comprising the medication.

27. A method for determining a state of a compression spring positioned between at least a portion of a first capacitive plate and at least a portion of a second capacitive plate, the method comprising: measuring, by a processing circuit, a temperature using a temperature sensor; measuring, by the processing circuit, a capacitance between the first capacitive plate and the second capacitive plate; and determining, by the processing circuit, whether the compression spring is in a compressed state or an expanded state based on the measured capacitance and the measured temperature.

28. The method of claim 27, wherein the compression spring is part of a medication-delivery device, and is configured to, after the medication-delivery device is activated to dispense medication, transition from a compressed state to an expanded state, or to transition from the expanded state to the compressed state.

29. The method of claim 28, wherein the medication-delivery device includes a syringe assembly that is movable between a storage position and an injection position, and the compression spring is configured to transition from the compressed state to the expanded state so as to move the syringe assembly from the injection position to the storage position.

30. The method of any of claims 28-29, further comprising reporting at least one of the measured capacitance, the measured temperature, and data indicative of whether the compression spring is in the compressed state or the expanded state to an external device via a wireless communication.

31. The method of any of claims 27-30, wherein determining whether the compression state is in the compressed state or the expanded state comprises: determining a capacitance threshold to apply based on the measured temperature; determining that the compression spring is in the compressed state when the measured capacitance is greater than the determined capacitance threshold; and determining that the compression spring is in the expanded state when the measured capacitance is less than the determined capacitance threshold.

32. The method of claim 31, wherein determining the capacitance threshold to apply comprises determining to apply a first capacitance threshold when the measured temperature is at a first temperature and to apply a second capacitance threshold that is lower than the first capacitance threshold when the measured temperature is below the first temperature.

1. A medication delivery device with a sensing system for determining a state of a compression spring of the medication delivery device, the sensing system comprising:
a first capacitive plate and a second capacitive plate configured such that the compression spring is disposed between at least a portion of the first capacitive plate and at least a portion of the second capacitive plate;
a temperature sensor; and
at least one processing circuit connected to the first capacitive plate, the second capacitive plate, and the temperature sensor, the at least one processing circuit configured to:
measure a capacitance between the first capacitive plate and the second capacitive plate,
measure a temperature based on a signal output by the temperature sensor, and
determine whether the compression spring is in a compressed state or an expanded state based on the measured capacitance and the measured temperature.

2. The medication delivery device of claim 1 wherein the at least one processing circuit comprises a first processing circuit at the medication delivery device and a second processing circuit at an external device, and wherein:
the first processing circuit is configured to measure the capacitance, measure the temperature, and communicate the measured capacitance and the measured temperature to the second processing circuit at the external device; and
the second processing circuit is configured to determine, based on the communicated capacitance and the communicated temperature, whether the compression spring is in the compressed state or the expanded state.

3. (canceled)

4. (canceled)

5. The medication delivery device of claim 1, wherein the sensing system is configured to receive harvested power wirelessly from an external power source.

6. The medication delivery device of claim 1, wherein the at least one processing circuit is configured to determine that the compression spring is in the compressed state when the measured capacitance is greater than a capacitance threshold, and to determine that the compression spring is in the expanded state when the measured capacitance is less than the capacitance threshold.

7. The medication delivery device of claim 6, wherein the at least one processing circuit is configured to adjust the capacitance threshold based on the measured temperature.

8. (canceled)

9. The medication delivery device of claim 1, wherein the first capacitive plate comprises a first plurality of fingers, the second capacitive plate comprises a second plurality of fingers, and the first capacitive plate and the second capacitive plate are disposed such that the first plurality of fingers is interleaved with the second plurality of fingers.

10. The medication delivery device of claim 1, wherein the at least one processing circuit is configured to determine whether a hand of a user is gripping the medication delivery device based on the measured capacitance.

11. The medication delivery device of claim 1, further comprising at least a third capacitive plate disposed on a user-graspable portion of the medication delivery device, wherein the at least one processing circuit is configured to determine whether a hand of a user is gripping the medication delivery device based on a capacitance measured by the third capacitive plate.

12. The medication delivery device of claim 1, further comprising a medication.

13. The medication delivery system of claim 1, further comprising:
a medication-delivery device housing;
a syringe assembly at least partially disposed within the medication-delivery device housing, the syringe assembly including a reservoir configured to hold a medication and an injection needle; and
a drive mechanism at least partially disposed within the medication-delivery device housing configured to, upon activation by a user, dispense the medication from the syringe assembly via the injection needle, wherein the drive mechanism includes the compression spring that is configured to transition from the compressed state to the expanded state, or to transition from the expanded state to the compressed state, after said activation.

14. (canceled)

15. (canceled)

16. (canceled)

17. (canceled)

18. (canceled)

19. The medication delivery system of claim 13, wherein the first capacitive plate, the second capacitive plate, the temperature sensor, and at least part of the at least one processing circuit are attached to a label secured to an external surface of the medication-delivery device housing.

20. The medication delivery system of claim 13, wherein:
the medication-delivery device housing defines an injection aperture;
the syringe assembly is movable between a storage position in which the injection needle does not extend through the injection aperture and an injection position in which the injection needle does extend through the injection aperture; and
the compression spring is configured to, after the medication-delivery device is activated, transition from the compressed state to the expanded state so as to move the syringe assembly from the injection position to the storage position after the medication has been dispensed.

21. The medication delivery system of claim 13, wherein the compression spring is configured to transition from the compressed state to the expanded state so as to dispense medication from the reservoir through the injection needle.

22. The medication delivery system of claim 13, further comprising a metallic shield that covers the first capacitive plate and the second capacitive plate.

23. (canceled)

24. (canceled)

25. (canceled)

26. (canceled)

27. A method for determining a state of a compression spring positioned between at least a portion of a first capacitive plate and at least a portion of a second capacitive plate, the method comprising:
measuring, by a processing circuit, a temperature using a temperature sensor;
measuring, by the processing circuit, a capacitance between the first capacitive plate and the second capacitive plate; and
determining, by the processing circuit, whether the compression spring is in a compressed state or an expanded state based on the measured capacitance and the measured temperature.

28. The method of claim 27, wherein the compression spring is part of a medication-delivery device, and is configured to, after the medication-delivery device is activated to dispense medication, transition from a compressed state to an expanded state, or to transition from the expanded state to the compressed state.

29. The method of claim 28, wherein the medication-delivery device includes a syringe assembly that is movable between a storage position and an injection position, and the compression spring is configured to transition from the compressed state to the expanded state so as to move the syringe assembly from the injection position to the storage position.

30. The method of claim 28, further comprising reporting at least one of the measured capacitance, the measured temperature, and data indicative of whether the compression spring is in the compressed state or the expanded state to an external device via a wireless communication.

31. The method of claim 27, wherein determining whether the compression state is in the compressed state or the expanded state comprises:
determining a capacitance threshold to apply based on the measured temperature;
determining that the compression spring is in the compressed state when the measured capacitance is greater than the determined capacitance threshold; and
determining that the compression spring is in the expanded state when the measured capacitance is less than the determined capacitance threshold.

32. The method of claim 31, wherein determining the capacitance threshold to apply comprises determining to apply a first capacitance threshold when the measured temperature is at a first temperature and to apply a second capacitance threshold that is lower than the first capacitance threshold when the measured temperature is below the first temperature.
